(19) United States
(12) Patent Application Publication
(10) Pub. No.: US 2024/0075180 A1
(43) Pub. Date: Mar. 7, 2024

(54) PLASMA GENERATION MODULE, PURIFICATION COMPONENT, DEVICE AND AIR CONDITIONING SYSTEM

(71) Applicants: GD MIDEA AIR-CONDITIONING EQUIPMENT CO., LTD., Foshan (CN); GD Midea Heating & Ventilating Equipment Co., Ltd., Foshan (CN); MIDEA GROUP (SHANGHAI) CO., LTD., Shanghai (CN); GUANGDONG MIDEA WHITE HOME APPLIANCE TECHNOLOGY INNOVATION CENTER CO., LTD., Foshan (CN); MIDEA GROUP CO., LTD., Foshan (CN)

(72) Inventors: Xianjie WANG, Foshan (CN); Fang LIU, Foshan (CN); Wenwen WANG, Foshan (CN); Ben LIU, Foshan (CN)

(21) Appl. No.: 18/389,542

(22) Filed: Nov. 14, 2023

Related U.S. Application Data
(63) Continuation of application No. PCT/CN2022/097411, filed on Jun. 7, 2022.

(30) Foreign Application Priority Data
May 18, 2021 (CN) 202110540576.7

(51) Int. Cl. A61L 9/18; H05H 1/24

(52) U.S. Cl. CPC A61L 9/18; H05H 1/2431; A61L 2209/14; A61L 2209/16

(57) ABSTRACT

A plasma generation module includes a first electrode that includes a linear structure of a flexible material; a second electrode having a structure made of a flexible material and is disposed opposite to the first electrode. The first electrode and the second electrode are configured to form a linear discharge region between the first electrode and the second electrode. The plasma generation module includes a dielectric layer provided between the first electrode and the second electrode.

### CROSS-REFERENCE TO RELATED APPLICATIONS

This application is a continuation of International (PCT) Patent Application No. PCT/CN2022/097411, filed on Jun. 7, 2022, which claims a priority of the Chinese patent application No. 202110540576.7, filed on May 18, 2021 and entitled “Plasma Generation Module, Purification Component, Device and Air Conditioning System”, the entire contents of which are incorporated herein by reference.

### TECHNICAL FIELD

The present disclosure relates to the field of purification technology, and in particular to a plasma generation module, a purification component, an air purification device and an air conditioning system.

### BACKGROUND

Plasma oxidation technology utilizes the energy and an active constituent generated by a discharge between two electrodes to eliminate VOC (volatile organic compounds), viruses and bacteria from the air, and decompose them into water and carbon dioxide. It has distinctive advantages over other air purification technologies.

A plasma can be generated by adopting two plate electrodes (one high voltage and one ground), and a layer of dielectric provided between the two plate electrodes. The layer of dielectric is configured to realize a dielectric barrier discharge (DBD). When plasma is utilized to eliminate virus and germ, air is made to flow through a discharge region between the two plate electrodes, so that organic contaminants in the air can be oxidized and the bacteria and viruses in the air can be killed by high-energy particles and an electric field generated by the plasma. The discharge between the two plate electrodes is a filamentary discharge, which may be independent of a surface size of electrode plates. For example, the discharge region formed may not be affected no matter how large the areas of the two plate electrodes are. Therefore, the plasma may have a small range of action and a shape of a plasma generation module may not be changed, and may not be suitable for a planar region or space having a larger area.

### SUMMARY

In one aspect of the disclosure, a plasma generation module is provided, including: a first electrode that includes a linear structure of flexible material; a second electrode, made of a flexible material and disposed opposite to the first electrode to form a linear discharge region between the first electrode and the second electrode; and a dielectric layer disposed between the first electrode and the second electrode.

The first electrode is a linear structure of flexible material, and the second electrode is disposed opposite to the first electrode and also made of flexible material, so that a linear discharge region can be formed between the first electrode and the second electrode. A dielectric layer is also provided between the first electrode and the second electrode. The discharge generated by the first electrode and the second electrode acts on the air, and therefore a dielectric barrier discharge in the linear region is realized and a linear and bendable plasma generation module is obtained. A length and a bending deformation of the plasma generation module can be set according to an area, space size and shape of an application scene. Therefore, in some embodiments, the plasma generation module may have a flexible and changeable modality, and can be applied to a planar region or space with a larger area, and further may be adapted to different application scenarios.

In some embodiments, a purification component includes the plasma generation module. A purification efficiency of the purification component may be improved by the above plasma generation module, and a structural design of the purification component is not limited by the structure of the plasma generation module.

In some embodiments, an air purification device includes the purification component. A modality and structure of the air purification device are not limited by the structure of the plasma generation module, so that the modality of the air purification device can be more diversified, and the purification efficiency of the air purification device can be improved.

In some embodiments, an air conditioning system includes the purification component such that a space occupied by the purification components in the air conditioning system may be reduced; an impact on the structural design of the air conditioning system may be reduced; and a purification efficiency of the air conditioning system may be improved.

### BRIEF DESCRIPTION OF DRAWINGS

In order to more clearly illustrate the technical solutions in embodiments of the present disclosure, a brief introduction will be made below to the accompanying drawings needed to be used in the description of the embodiments. Obviously, the accompanying drawings in the following description are some embodiments of the present disclosure. For those of ordinary skill in the art, other accompanying drawings can be obtained based on these accompanying drawings without creative efforts.

FIG. 1 shows a first structural schematic diagram of a plasma generation module according to some embodiments of the present disclosure;

FIG. 2 is a schematic cross-sectional view of the plasma generation module in FIG. 1;

FIG. 3 shows a second structural schematic diagram of the plasma generation module according to some embodiments of the present disclosure;

FIG. 4 shows a third structural schematic diagram of the plasma generation module according to some embodiments of the present disclosure;

FIG. 5 shows a fourth structural schematic diagram of the plasma generation module according to some embodiments of the present disclosure;

FIG. 6 shows a fifth structural schematic diagram of the plasma generation module according to some embodiments of the present disclosure;

FIG. 7 shows a sixth structural schematic diagram of the plasma generation module according to some embodiments of the present disclosure;

FIG. 8 shows a seventh structural schematic diagram of the plasma generation module according to some embodiments of the present disclosure;

FIG. 9 shows an eighth structural schematic diagram of the plasma generation module according to some embodiments of the present disclosure;

FIG. 10 shows a first planar configuration of the plasma generation module of a purification component according to some embodiments of the present disclosure;

FIG. 11 shows a second planar configuration of the plasma generation module of the purification component according to some embodiments of the present disclosure;

FIG. 12 shows a third planar configuration of the plasma generation module of the purification component according to some embodiments of the present disclosure;

FIG. 13 to FIG. 18 show schematic diagrams of the plasma generation module of the purification component according to embodiments of the present disclosure disposed on a carrier member; and

FIG. 19 shows a schematic diagram of a flow of air in the purification component.

### REFERENCE SIGNS

plasma generation module 10, first electrode 11, second electrode 12, dielectric layer 13, catalyst layer 14, filtering passage 20, and carrier member 30.

### DESCRIPTION OF EMBODIMENTS

The technical solutions in the embodiments of the disclosure will be clearly and completely described below in conjunction with the accompanying drawings in the embodiments of the disclosure. Obviously, the described embodiments are only some embodiments of the disclosure, not all embodiments. Based on the embodiments in this disclosure, all other embodiments obtained by those of ordinary skill in the art without making creative efforts fall within the protection scope sought for by the present disclosure.

It should be noted that all directional indications in the embodiments of the present disclosure are only used to explain the relative positional relationship, a stage of movement, etc. among various components in a specific posture. If the specific posture changes, the directional instructions will be changed accordingly.

In this disclosure, unless otherwise explicitly stated and defined, the terms “connect”, “fix”, etc. should be understood in a broad sense. For example, the “fix” can be a fixed connection, a detachable connection, or an integral body. The “connect” can be a mechanical connection, or an electrical connection; can be a direct connection or an indirect connection through an intermediate medium; or can be an internal communication between two elements or an interactive relationship between two elements, unless otherwise clearly defined. For those of ordinary skill in the art, the specific meanings of the above terms in this disclosure can be understood according to specific circumstances.

In addition, if there are descriptions involving “first”, “second” etc. in the present disclosure, the descriptions of “first”, “second”, etc. are used only for a purpose of description and shall not be understood to indicate or imply their relative importance, or designate implicitly the number of technical features indicated. Therefore, the features defined with “first” and “second” may explicitly or implicitly include at least one of the features. In addition, the technical solutions of the various embodiments can be combined with each other, but a technical solution combined by technical solutions of the various embodiments shall be capable of being realized by those skilled in the art. When a combination of technical solutions is contradictory or cannot be realized, it should be considered that the combination of technical solutions does not exist, and is not within a scope of protection sought for by the present disclosure.

A plasma generation module 10 according to the embodiments of the present disclosure is used to utilize high-energy particles and electric fields generated by plasma to oxidize VOC and other organic contaminants and kill bacteria and viruses. Thus, the plasma generation module 10 can be used to purify air and other gases, or can also be used to purify some non-conductive liquids.

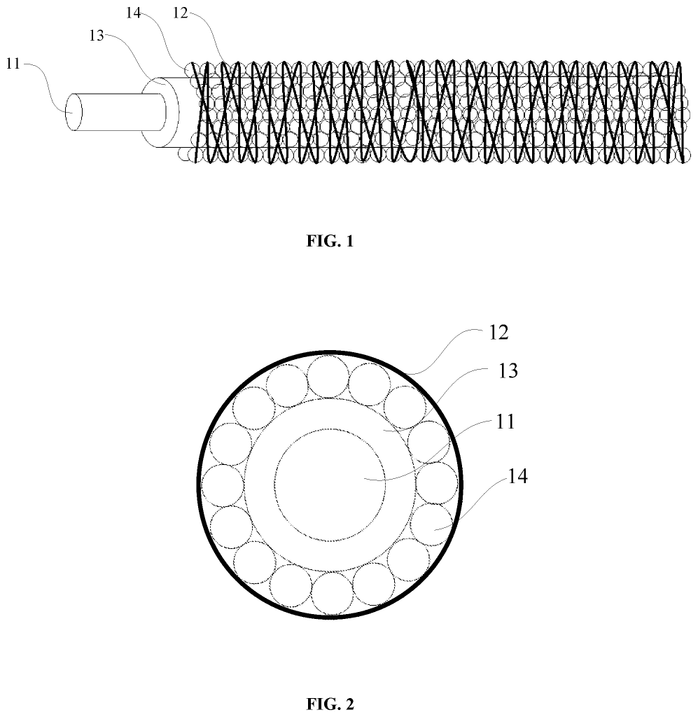

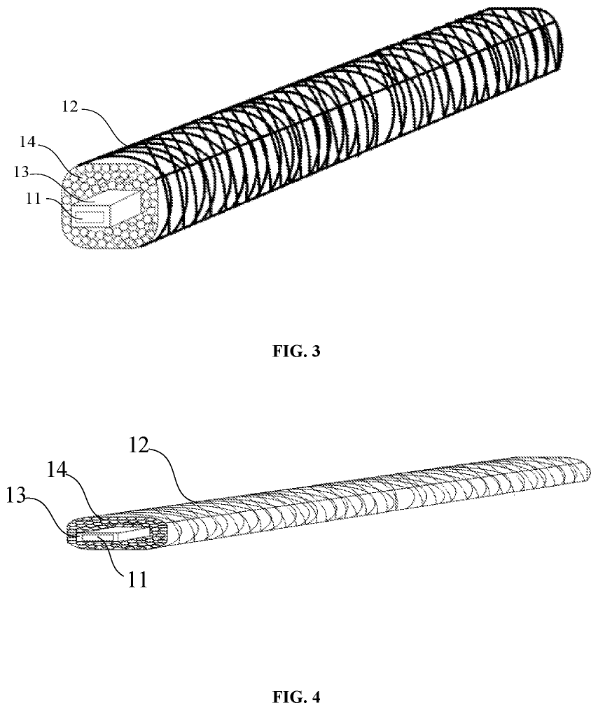

Referring to FIG. 1 to FIG. 6, a plasma generation module 10 according to some embodiments of the present disclosure may include: a first electrode 11, a second electrode 12 and a dielectric layer 13. The first electrode 11 is a linear structure made of flexible material. The second electrode 12 is also made of flexible material, and the first electrode 11 and the second electrode 12 are disposed oppositely. A linear discharge region, by oppositely disposed the second electrode 12 and the first electrode 11, can be formed between the first electrode 11 and the second electrode 12. The dielectric layer 13 is disposed between the first electrode 11 and the second electrode 12 to enable a formation of a dielectric barrier discharge between the first electrode 11 and the second electrode 12.

In some embodiments of the present disclosure, the linear discharge region indicates that the discharge region between the first electrode 11 and the second electrode 12 is distributed linearly, and a length dimension of the discharge region is a first preset multiple of a discharge distance, the first preset multiple being a value greater than 1, the discharge distance being a distance between the first electrode 11 and the second electrode 12.

It should be understood that when an area of an application location of the plasma generation module 10 is larger, the length dimension of the discharge region is larger. In practical applications, when the plasma generation module 10 is used in a larger area or space, a length of the discharge region is much larger than the discharge distance. In some embodiments, the length dimension of the discharge region may be dozens, hundreds, or even more multiples of the discharge distance, and the length of the discharge region changes correspondingly with a length of the first electrode 11. If the first electrode 11 is arranged in a curved shape, the discharge region will be distributed in a curved manner accordingly.

Either one of the first electrode 11 and the second electrode 12 is used to connect to a power source to provide an operating voltage, and the other electrode is grounded or connected to a voltage lower than the operating voltage. When the power source supplies power to either one of the first electrode 11 and the second electrode 12, a voltage difference will be generated between the first electrode 11 and the second electrode 12, so that the dielectric barrier discharge will be formed between the first electrode 11 and the second electrode 12. The dielectric barrier discharge acts on a fluid (such as air, exhaust gas or liquid from certain processes) flowing between the first electrode 11 and the second electrode 12, to cause the fluid and substances in the fluid to be charged into a plasma state to generate a plasma. The generated plasma can eliminate VOC, viruses and bacteria in the fluid, and decompose them into water and carbon dioxide, thereby achieving a purification effect without secondary pollution to the environment.

Since the first electrode 11 is of the linear structure, and the second electrode 12 is disposed opposite to the first electrode 11, a linear plasma generation module 10 thus formed can be increased in length according to an application scenario, so that a total discharge length is increased. In addition, since the first electrode 11 and the second electrode 12 are both made of flexible materials, the formed plasma generation module 10 has linear and bendable characteristics, thereby achieving bending deformation, that is, the plasma generation module 10 can be bent and coiled to act on planar or spatial structures of different sizes and shapes according to different needs. Furthermore, the discharge region between the two electrodes can be expanded from a local single point to a planar region or even a spatial structure to be applicable for different scenarios.

In some embodiments of the present disclosure, the first electrode 11 being a linear structure of flexible material can be achieved from structural and material perspectives. From the material perspective, a conductive metal that can be bent and deformed can be selected to be made into a metal wire or a metal stripe to achieve a linear structure of flexible material. From the structural perspective, it can be selected to dispose a conductor layer on other flexible materials, such as a silicone or Polytetrafluoroethylene of a linear structure on which a graphite layer or a metal layer is deposited, to achieve the linear structure of flexible material.

In some embodiments of the present disclosure, the linear structure of the first electrode 11 is for the length of the first electrode 11. The length dimension of the first electrode 11 is a second preset multiple of a cross-sectional dimension of the first electrode 11, the second preset multiple being a value greater than 1.

It should be understood that, when the area of the application location of the plasma generation module 10 is larger, the length dimension of the first electrode 11 is larger, and the discharge region formed between the first electrode 11 and the second electrode 12 is longer, and thus a range of action of plasma is greater. In practical applications, for the plasma generation module 10 used in a larger planar region or a larger space, the length dimension of the first electrode 11 is much larger than the cross-sectional dimension thereof. However, the specific length dimension of the first electrode 11 can be set according to a size of an applied planar region or space. While the cross-sectional dimension of the first electrode 11 can be constant and does not need to be changed according to a different size of the applied planar region or space.

It can be understood that the length of the first electrode 11 is positively related to a size of a location where the plasma generation module 10 is disposed. That is, the larger the location where the plasma generation module 10 needs to be located is, the longer the plasma generation module 10 is, and the larger the length dimension of the first electrode 11 is, while the cross-sectional dimension of the first electrode 11 is independent of the size of this location. In actual application scenarios, the length dimension of the first electrode 11 may be dozens, hundreds, or even more multiples of the cross-sectional dimension thereof.

It can be understood that the length of the second electrode 12 is adapted to the length of the first electrode 11. The length of the second electrode 12 may be the same as the length of the first electrode 11, or the length of the second electrode 12 may be greater than the length of the first electrode 11, so as to form a curved second electrode 12 which is disposed opposite to the first electrode 11. For example, the first electrode 11 may be a metal wire or metal stripe made of copper, tungsten or iron, so as to achieve the bendable and linear characteristics of the first electrode 11. The second electrode 12 also may be a tinsel or metal film made of copper, tungsten or iron, so as to achieve the bendable characteristic of the second electrode 12.

In some embodiments, an airflow passage may be opened on the second electrode 12 so that the discharge region between the first electrode 11 and the second electrode 12 can be in contact with an external fluid, thus the plasma can directly eliminate the contaminants in the fluid and the produced active substances can be diffused into the surrounding fluid for action, and in turn the range of action of plasma is not only limited to between the two electrodes. A resistance endured by a filtered fluid when the filtered fluid passes through the discharge region between the first electrode 11 and the second electrode may be also reduced by the airflow passage opened on the second electrode 12.

In some embodiments of the present disclosure, the second electrode 12 opened with the airflow passage can be implemented in a variety of ways.

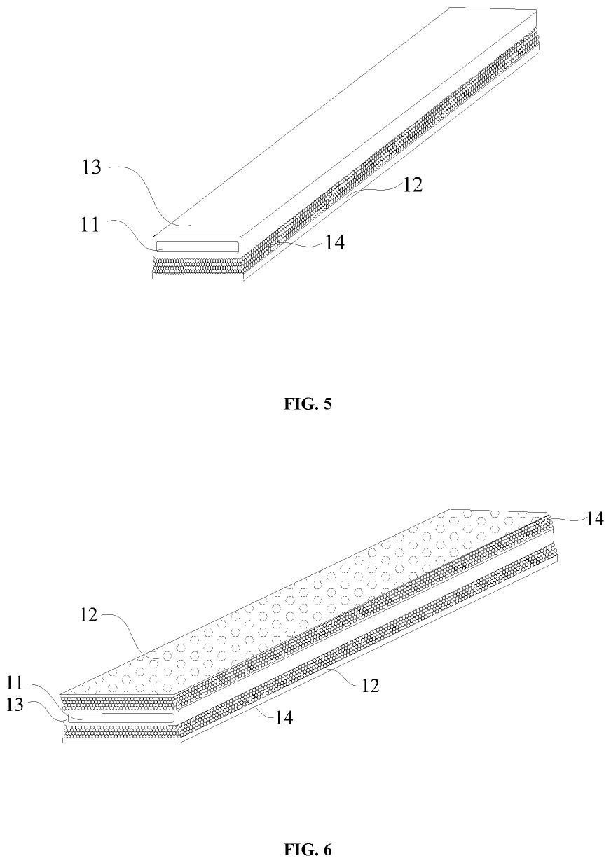

Referring to FIG. 1, the second electrode 12 may be an integrally formed metal mesh structure, so that the external fluid can pass through meshes of the metal mesh structure to contact the discharge region. Referring to FIG. 6, the second electrode 12 may be a porous metal plate structure, so that the external fluid can contact the discharge region through plate holes of the porous metal plate structure.

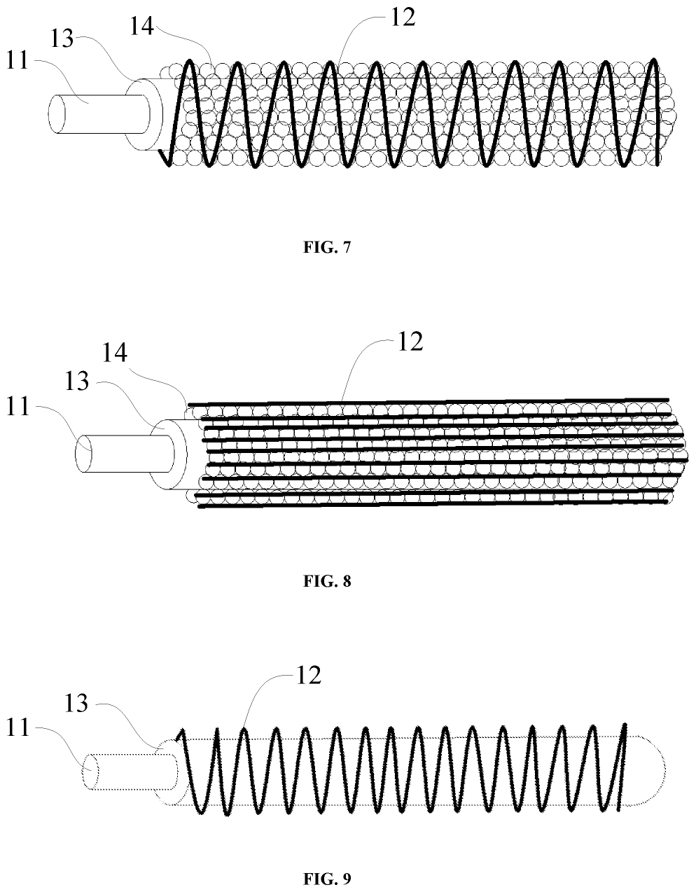

The second electrode 12 may also be one or more metal wires wound around the first electrode 11. For example, as shown in FIG. 7, the second electrode 12 is spirally wound around the first electrode 11. In some embodiments, the second electrode 12 may be wound around the first electrode 11 in other curved shapes. Referring to FIG. 8, the second electrode 12 may be a plurality of metal wires disposed around the first electrode 11 at an interval so that the external fluid can contact the discharge region through gaps between the metal wires. For example, various metal wires of the second electrode 12 may be parallel or have a certain angle therebetween.

The above-mentioned structures each realize the airflow passage of the second electrode 12, and therefore a region of action of plasma can be significantly increased, and in turn a plasma effect can be increased, and a structure of the second electrode 12 is also simplified at the same time.

An arrangement of the second electrode 12 relative to other components is depending on a position of the dielectric layer 13 and whether the catalyst layer 14 is provided, that is, there are different arrangements of the second electrode 12 relative to other components according to different positions of the dielectric layer 13 and whether the catalyst layer 14 is provided.

In some application scenarios, the catalyst layer 14 may not be needed. For example, in a scenario of purifying liquid, no catalyst layer 14 is required, since a reaction mode between the active substances and molecules of the contaminants is a surface reaction. For another example, in some industrial scenarios that a decomposition of ozone does not need to be considered, and therefore the like, the catalyst layer 14 can be omitted.

Referring to FIG. 9, if the catalyst layer 14 is not provided in the plasma generation module 10, the dielectric layer 13 may be wrapped on a side wall of the first electrode 11, and then the second electrode 12 may be wrapped on an outside of the dielectric layer 13, thereby forming the plasma generation module 10 with a three-layer wrapping structure. A flexibility of the plasma generation module 10 is ensured by this three-layer wrapping structure, so that the plasma generation module 10 can be bent and deformed. In some embodiments, quartz or ceramic particles may also be selected to be formed on a flexible carrier, or the dielectric layer 13 in granular state may be directly used to be wrapped by the second electrode 12, so that the dielectric layer 13 in granular state can be fixed in the second electrode 12 and the flexibility of the plasma generation module 10 is maintained.

If the catalyst layer 14 is not provided in the plasma generation module 10, the dielectric layer 13 may also be wrapped on a side wall of the first electrode 11, and the second electrode 12 is stacked on the first electrode 11 after being wrapped with the dielectric layer 13. In a stacked mode, the dielectric layer 13 is made of a flexible material, and the flexible material may be silicone or polytetrafluoroethylene, and therefore the flexibility of the plasma generation module 10 is ensured, so that the plasma generation module 10 can be bent and deformed.

The plasma generation module 10 according to some embodiments of the present disclosure may also include a catalyst layer 14. In scenarios that include the catalyst layer, the catalyst layer 14 may be disposed between the first electrode 11 and the dielectric layer 13, or between the dielectric layer 13 and the second electrode 12. An active constituent generated by the plasma generation module 10 contains ozone, and if the ozone overflows into the air, it will cause secondary pollution to the air. Therefore, surface sites are provided to promote the decomposition of the ozone and reduce the amount of residual ozone by the catalyst layer 14 between the first electrode 11 and the second electrode 12.

Referring to FIG. 1 to FIG. 4, the catalyst layer 14 may be disposed as follows: the dielectric layer 13 is wrapped on the side wall of the first electrode 11; the catalyst layer 14 is wrapped on an outside of the dielectric layer 13; and the second electrode 12 is wrapped on an outside of the catalyst layer 14, thereby forming the plasma generation module 10 with a four-layer wrapping structure. In some embodiments, a structural stability of the plasma generation module 10 may be improved through the four-layer wrapping structure.

In some embodiments, in the four-layer wrapping structure of the plasma generation module 10, the catalyst layer 14 may be a separate layer relative to the dielectric layer 13, and catalyst particles are directly fixed to an interior of the second electrode 12, and therefore an attachment substrate of the catalyst particles can be omitted. In some embodiments, the catalyst layer 14 may also be a layer of substrate with catalyst particles attached thereon. In some embodiments, the catalyst layer 14 may also be catalyst particles directly attached to the dielectric layer 13, integrating the catalyst layer 14 and the dielectric layer 13.

In the four-layer wrapping structure, the first electrode 11 may be a metal wire whose cross section is circular, oval or square, and therefore it is convenient for the wrapping of the dielectric layer 13, the catalyst layer 14 and the second electrode 12 layer by layer, and the structural stability may be further improved. In some embodiments, a shape of the first electrode 11 is any shape suitable for wrapping the catalyst layer 14, and the cross section of the first electrode 11 is not limited to the shapes listed above.

In some embodiments, the dielectric layer 13 may include: a first sub-dielectric layer disposed on an outer surface of the first electrode 11 and a second sub-dielectric layer disposed on an outer surface of the second electrode 12. With such compact design, an extending of length can be allowed and at the same time uniform discharge distances between the two electrodes are not changed, and a multi-layer insulation protection may be achieved to improve safety.

In some embodiments, the first sub-dielectric layer 13 may be sleeved on the first electrode 11 or coated on the first electrode 11. The second sub-dielectric layer may be coated on each metal wire used to form the second electrode 12, or sleeved on each metal wire used to form the second electrode 12. For example, if the second electrode 12 is a structure of porous metal plate, the second sub-dielectric layer may be coated on a plate body portion of the porous metal plate while plate hole areas of the porous metal plate are exposed, so that the fluid can pass through the plate hole areas of the porous metal plate to contact with the discharge region between the two electrodes. To improve the safety of electricity use, the dielectric layer 13 may be wrapped at least on the electrode for connecting to the power source.

If the dielectric layer 13 includes a first sub-dielectric layer disposed on the outer surface of the first electrode 11 and a second sub-dielectric layer disposed on the outer surface of the second electrode 12, the catalyst layer 14 may be disposed between the first sub-dielectric layer and the second sub-dielectric layer.

If the dielectric layer 13 is only disposed on the outer surface of the second electrode 12, the catalyst layer 14 may also be directly wrapped on the side wall of the first electrode 11, and the second electrode 12 provided with the dielectric layer 13 is wrapped on an outside of the catalyst layer 14. Therefore a multi-layer wrapping structure can also be formed, and the structural stability of the plasma generation module 10 may be further improved.

In some embodiments of the present disclosure, the first electrode 11 is a high-voltage electrode, and used to connect to the power source. For example, the high voltage may be about 8 kV and the voltage used will be different depending on the actual situation. The second electrode 12 is grounded. The high-voltage electrode is wrapped inside by the catalyst layer 14 and the low-voltage second electrode 12, improving the safety of electricity use. The first electrode 11 may also be grounded as a low-voltage electrode, and the second electrode 12 can be used as a high-voltage electrode for connecting to the power source.

Referring to FIG. 5 and FIG. 6, the dielectric layer 13, the catalyst layer 14 and the second electrode 12 may be stacked. In such stacked arrangement, the first electrode 11 may be a flat and stripe-shaped electrode, and the dielectric layer 13 may be directly wrapped on the first electrode 11. As shown in FIG. 5, a catalyst layer 14 and a second electrode 12 may be in sequence stacked on one surface of the first electrode 11 wrapped with the dielectric layer 13. As shown in FIG. 6, the catalyst layer 14 and the second electrode 12 may be stacked on both surfaces of the first electrode 11 wrapped with the dielectric layer 13, so that two mutually separated linear discharge regions are formed between the first electrode 11 and the two second electrodes 12.

In some embodiments, when that the dielectric layer 13 is not provided on the first electrode 11, the catalyst layer 14 and the second electrode 12 may be directly stacked on at least one side wall of the flat and stripe-shaped first electrode 11.

If the dielectric layer 13, the catalyst layer 14 and the second electrode 12 are stacked, the external fluid can contact the discharge region from a gap between the first electrode 11 and the second electrode 12. Therefore, there is no need for opening an airflow passage on the second electrode 12, that is, the second electrode 12 can be a non-porous metal plate made of flexible material.

Through the above methods, the catalyst layer 14 can be fixed on the first electrode 11 by the second electrode 12, so that there is no gap both between the catalyst layer 14 and the first electrode 11, and between the catalyst layer 14 and the second electrode 12. With such compact design, the length of the plasma generation module 10 may be extended without changing the uniform discharge distances between the two electrodes. Moreover, an action of plasma can keep the coupled catalyst layer 14 in a state of continuous regeneration, thus there is no need to replace the catalyst. After the plasma generation module 10 is added to other ordinary filter screens (such as activated carbon filter screens), since most of organic molecules have been oxidized by the plasma generation module 10, a service life of the plasma generation module 10 is extended.

It should be understood that different materials selected for the catalyst layer 14 will play different roles in the plasma generation module 10. Next, a material selection for the catalyst layer 14 is described.

In some embodiments, the catalyst particles in the catalyst layer 14 may be metal oxide-based catalysts, thus the discharge is enhanced and the discharge distance between the first electrode 11 and the second electrode 12 can be shorten. In some embodiments, compared to a situation without catalyst layer 14, a maximum voltage value required for discharge between the first electrode 11 and the second electrode 12 may be reduced due to the added the catalyst layer 14.

In some embodiments, the metal oxide-based catalyst may be a porous metal oxide-based catalyst, and therefore not only the discharge distance can be shorten, and but also VOC molecules and viruses and bacteria in the air can be adsorbed on a surface of the porous metal oxide-based catalyst, so that a reaction mode of the active substances and the contaminants molecules changes from a gas phase reaction to a surface reaction. A concentration of surface contaminants is significantly higher than a concentration of gaseous contaminants, thus a reaction probability and an oxidation efficiency are increased, facilitating the improvement of a purification effect of air.

The catalyst particles in the catalyst layer 14 may be one metal oxide-based catalyst or a mixture of several metal oxide-based catalysts such as activated alumina, cerium oxide, molecular sieves, etc., and may have a spherical, columnar or other shape. Different catalyst particles may be selected according to different application scenarios to adapt to different application scenarios. Particles in the catalyst layer 14 may also be formed by mixing and extruding one or more selected from the group consisting of activated carbon, molecular sieves and metal-organic framework (MOF), and may also contain precious metals Pt, Ba, metal oxides of Mn₂O_{X}, CuO, CeO, etc.

In order to increase a total discharge length while still obtaining uniform and stable discharge, the discharge distances between the first electrode 11 and the second electrode 12 are kept equal.

In order to keep the discharge distances between the first electrode 11 and the second electrode 12 uniform, a dimension of the first electrode 11 is uniform; thicknesses of various positions of the dielectric layer 13 are consistent; thicknesses of various positions of the catalyst layer 14 are consistent; and distributions and sizes of the catalyst particles of the catalyst layer 14 are uniform. Therefore, a shortest distance of any sections between the two electrodes are equal, so that the discharge distances are uniform, and a uniform and stable discharge may be more easily obtained.

Based on some embodiments of the present disclosure, a linear and bendable plasma generation module 10 is obtained, a modality of the plasma generation module 10 can be changed according to requirements of different scenarios. The fluid only needs to flow through a range of action of the plasma generation module 10, and the contaminants in the fluid can be eliminated by the plasma generation module 10.

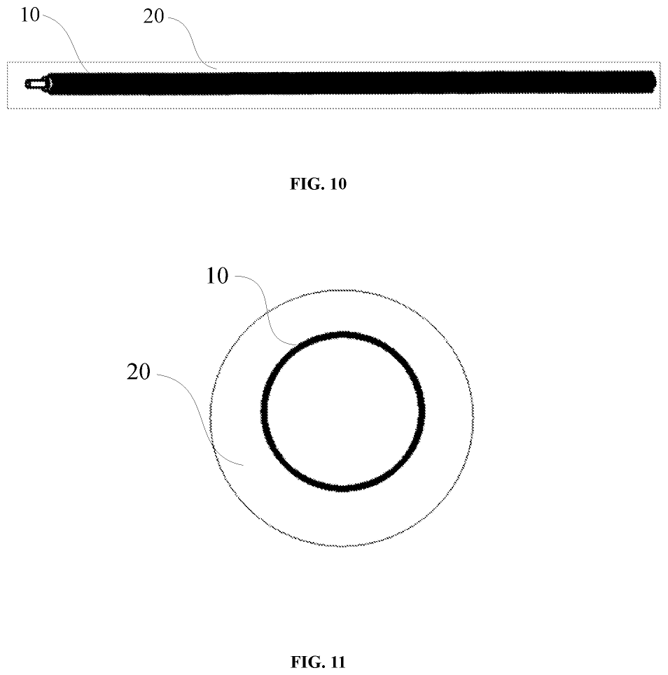

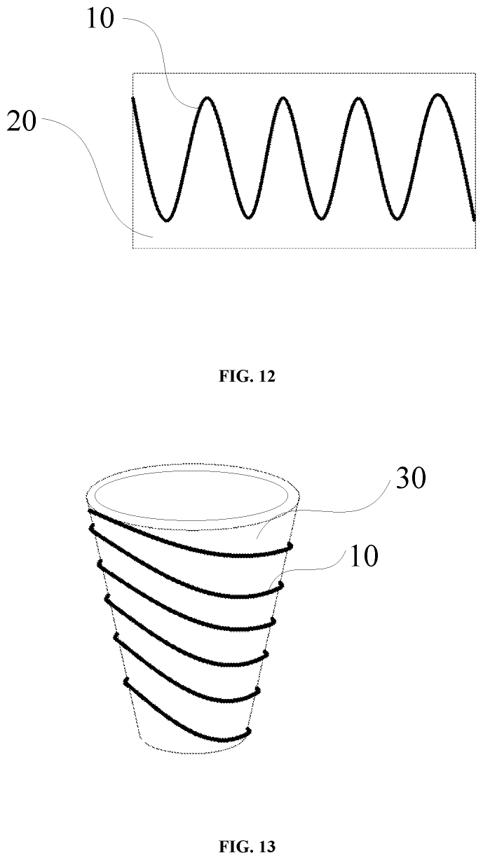

Referring to FIG. 10 to FIG. 13, configurations of the linear and bendable plasma generation module 10 formed by embodiments of the present disclosure can be changed according to application scenarios to adapt to planes or spaces of different sizes and shapes.

Next, a purification component provided by some embodiments of the present disclosure is described, including any one of the above-mentioned plasma generation modules 10. The purification component can be a filter element for air purification, a filter element for industrial gas purification, or a filter element for non-conductive liquid purification.

A purification component which can be suitable for different application scenarios can be formed by utilizing the above-mentioned plasma generation module 10. A structure of the formed purification component is no longer limited by a structure of the plasma generation module 10. A dimension of the purification component can also be reduced according to application scenarios, and an effective service time of the purification component is longer.

In some embodiments, the purification component may further include a filtering passage 20, and the plasma generation module 10 is disposed in the filtering passage 20. The plasma generation module 10 has a planar configuration adapted to a shape of cross section of the filtering passage 20. A length of the plasma generation module 10 can be selected according to the filtering passage 20, as long as the plasma generation module can be bent into a state which is adapted to the shape of cross section of filter passage 20.

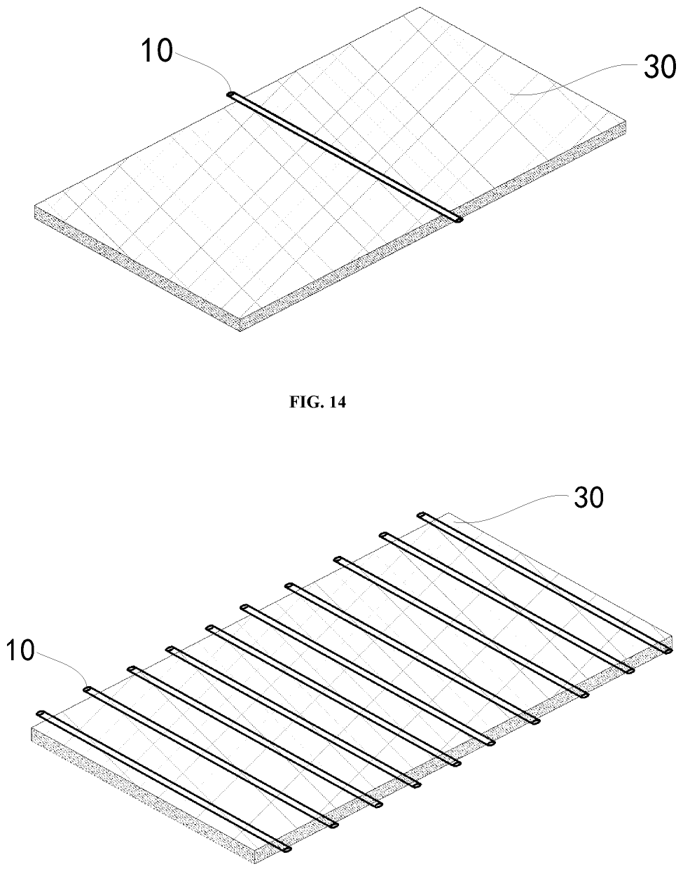

As shown in FIG. 10, the cross section of the filtering passage 20 is a slit, and the plasma generation module 10 is disposed within the filtering passage 20 in a stripe shape. Referring to FIG. 11, the cross section of the filtering passage 20 is circular, and the plasma generation module 10 may be bent into a ring shape to be disposed within the filtering passage 20. Referring to FIG. 12, the cross section of the filtering passage 20 is square, and the plasma generation module 10 may be in a curved shape, and may be crookedly disposed within the filtering passage 20 in a spiral shape, or may be crookedly disposed within the filtering passage 20 in a wavy shape.

The filtering passage 20 may be an air duct or other passage. One or more plasma generation modules 10 adapted to the shape of cross section of the filtering passage 20 may be disposed in the filtering passage 20. For example, the plasma generation modules 10 may be arranged at different positions of the filtering passage 20 according to shapes of these positions.

Referring to FIG. 13 to FIG. 18, the purification component may also include a carrier member 30. The plasma generation module 10 is disposed on the carrier member 30. In some embodiments, the carrier member 30 includes filter holes 31 to facilitate a fluid to reach a location where the plasma can act on. The carrier member 30 may be in contact with part or all segments of the plasma generation module 10, or the plasma generation module 10 may not be in contact with the carrier member 30, and therefore the range of action of plasma can be further increased.

In some embodiments, referring to FIG. 13, the carrier member 30 may be a three-dimensional structure, such as a hairdryer. For the carrier member 30 with a three-dimensional structure, a linear plasma generation module 10 may be disposed on an outer surface and/or an inner surface of a side wall of the carrier member 30. A way of arrangement may be that: one or more plasma generation modules 10 are wound around the outer surface and/or the inner surface of the side wall of the carrier member 30, or a plurality of plasma generation modules 10 are disposed at an interval on the outer surface and/or the inner surface of the side wall of the carrier member 30, so that the plasma generation module 10 is formed with a spatial configuration adapted to a spatial shape of the carrier member 30.

In other embodiments, the difference from the above embodiments is that the carrier member 30 may also be a planar structure. Referring to FIG. 14 to FIG. 18, one or more linear plasma generation module 10 are disposed on at least one surface of the carrier member 30. A planar shape of the carrier member 30 may be square, stripe, circular or irregular, and the plasma generation module 10 may be disposed on the surface of the carrier member 30 in a ring shape, a curved shape or a stripe shape.

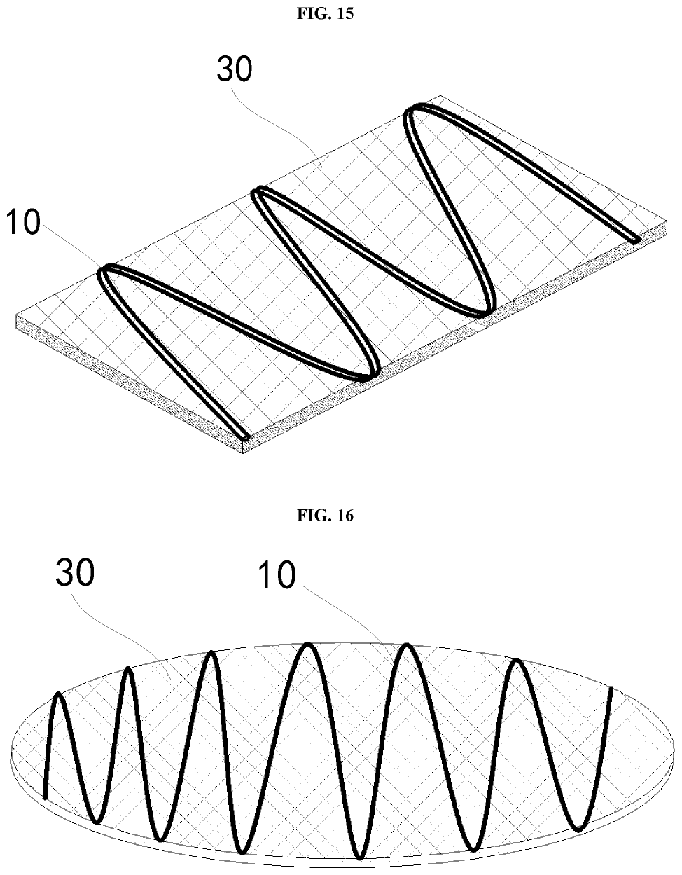

Referring to FIG. 14, one linear plasma generation module 10 may be disposed on the surface of the carrier member 30. Referring to FIG. 15, a plurality of linear plasma generation modules 10 may be disposed at an interval on the surface of the carrier member 30. Referring to FIG. 16, one or more linear plasma generation modules 10 may be disposed in a curved shape on the surface of the carrier member 30.

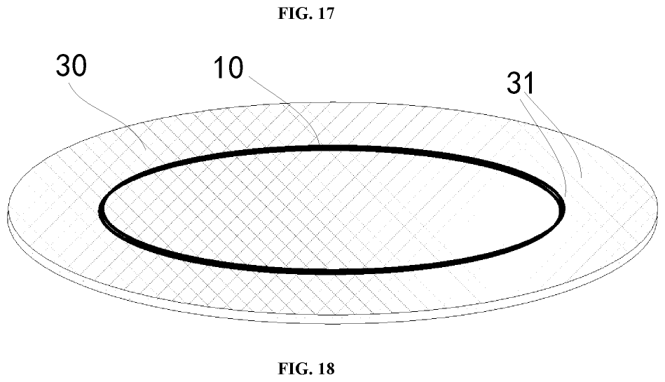

Referring to FIG. 17, if the planar shape of the carrier member 30 is circular, one or more linear plasma generation modules 10 may be disposed in a curved shape on a circular surface area of the carrier member 30. Referring to FIG. 18, one or more plasma generation modules 10 in the ring shape may be disposed on the circular surface area of the carrier member 30. If a plurality of annular plasma generation modules 10 are disposed in the circular surface area, various plasma generation modules 10 may be distributed concentrically.

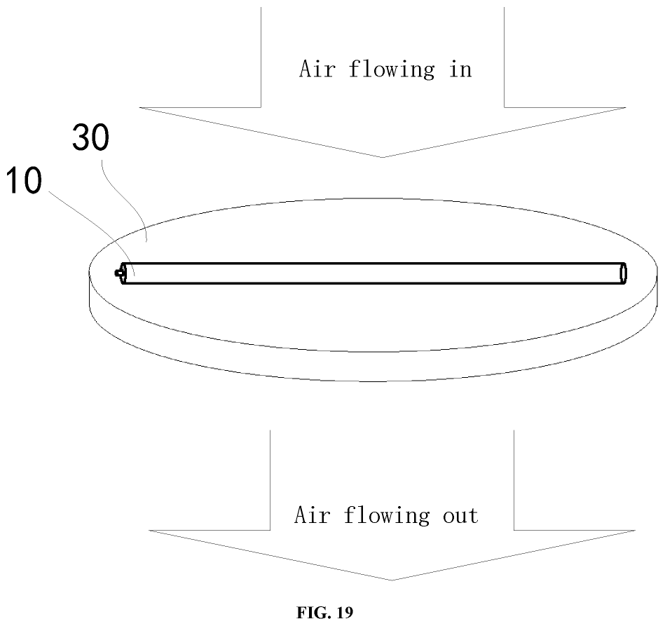

In embodiments of the present disclosure, the carrier member 30 may be a catalytic filter screen in which catalyst particles are contained. It can be understood that, under the circumstance that the carrier member 30 is the catalytic filter screen, the catalyst layer 14 may not be included in the plasma generation module 10, and the plasma generation module 10 may have a structure as shown in FIG. 9, thereby reducing a redundancy structure and costs. The shape of the plasma generation module 10 disposed on the catalytic filter screen and the number of the plasma generation modules 10 may be changed according to the application scenarios. If the carrier member 30 is the catalytic filter screen, as shown in FIG. 19, the air can enter from one side screen surface of the catalytic filter screen and then be purified under a joint action of the plasma generation module 10 and the catalytic filter screen, and then a purified air can flow out from another side screen surface of the catalytic filter screen.

An air purification device according to embodiments of the present disclosure is described below, including any of the above-mentioned purification components. The air purification device may be air purifiers, fresh air fans and other products.

Through the air purification device including the purification components described in any of the above embodiments, an action area of the air purification device is increased, thus a purification efficiency is improved. Moreover, the modality and structure of the air purification device are not limited by the plasma generation module 10, so that the modality and structure of the air purification device can be more diverse.

Below, an air conditioning system according to embodiments of the present disclosure is described, including any of the above-mentioned purification components. The air conditioning system may be a central air conditioning, household air conditioning, etc. The modality of the plasma generation module 10 within the air conditioning system can be changed to adapt to a space in the air conditioning system, and thus the space occupied in the air conditioning system is reduced.

In the description of this specification, reference to the terms “one embodiment”, “some embodiments”, “an example”, “specific examples”, or “some examples” or the like indicates that specific features, structures, materials or characteristics described in connection with the embodiment or example are included in at least one embodiment or example of the present disclosure. In this specification, the schematic expressions of the above terms are not necessarily directed to the same embodiment or example. Furthermore, the specific features, structures, materials or characteristics described may be combined in any suitable manner in any one or more embodiments or examples. Furthermore, those skilled in the art may join and combine the different embodiments or examples described in this specification.

1. A plasma generation module, comprising:
a first electrode comprising a linear structure of a flexible material;
a second electrode comprising a flexible material and disposed opposite to the first electrode, wherein the first electrode and the second electrode are configured to form a linear discharge region between the first electrode and the second electrode; and
a dielectric layer disposed between the first electrode and the second electrode.

2. The plasma generation module according to claim 1, wherein an airflow passage is opened on the second electrode.

3. The plasma generation module according to claim 2, wherein the second electrode is an element selected from the group consisting of an integrally formed metal mesh structure, a porous metal plate structure, one or more metal wires wound around the first electrode, and a plurality of metal wires disposed at an interval around the first electrode.

4. The plasma generation module according to claim 3, further comprising a catalyst layer, wherein the dielectric layer is wrapped on a side wall of the first electrode; the catalyst layer is wrapped on an outside of the dielectric layer; and the second electrode is wrapped on an outside of the catalyst layer.

5. The plasma generation module according to claim 4, wherein the first electrode is a metal wire whose cross section is circular, oval or square.

6. The plasma generation module according to claim 1, further comprising a catalyst layer, wherein the dielectric layer, the catalyst layer and the second electrode are stacked.

7. The plasma generation module according to claim 6, wherein the first electrode is flat and stripe-shaped.

8. The plasma generation module according to claim 1, further comprising a catalyst layer disposed between the first electrode and the dielectric layer, or between the dielectric layer and the second electrode.

9. The plasma generation module according to claim 1, wherein the dielectric layer comprises:
a first sub-dielectric layer, disposed on an outer surface of the first electrode; and
a second sub-dielectric layer, disposed on an outer surface of the second electrode;
and wherein the plasma generation module further comprises a catalyst layer disposed between the first sub-dielectric layer and the second sub-dielectric layer.

10. The plasma generation module according to claim 1, wherein discharge distances between the first electrode and the second electrode are equal.

11. An air purification device, comprising:
a purification component, comprising:
a plasma generation module comprising:
a first electrode, comprising a linear structure of a flexible material;
a second electrode, made of a flexible material and disposed opposite to the first electrode to form a linear discharge region between the first electrode and the second electrode; and
a dielectric layer, disposed between the first electrode and the second electrode.

12. The air purification device according to claim 11, further comprising a filtering passage, wherein the plasma generation module is disposed within the filtering passage.

13. The air purification device according to claim 12, wherein a cross section of the filtering passage is a slit, and the plasma generation module is in a stripe shape; or
the cross section of the filtering passage is circular, and the plasma generation module is in a ring shape; or
the cross section of the filtering passage is square, and the plasma generation module is in a curved shape.

14. The air purification device according to claim 11, further comprising a carrier member, wherein filter holes are opened on a side wall of the carrier member, and the plasma generation module is disposed on the carrier member.

15. The air purification device according to claim 14, wherein:
the carrier member is a three-dimensional structure, and the plasma generation module is wound around an outer surface and/or an inner surface of the side wall of the carrier member; or
the carrier member is a planar structure, and one or more plasma generation modules are disposed on at least one surface of the carrier member, wherein the plasma generation modules are disposed on the surface of the carrier member in a ring shape, a curved shape, or a stripe shape.

16. The air purification device according to claim 15, wherein the carrier member is a catalytic filter screen.

17. An air conditioning system, comprising: a purification component according to claim 11.

18. The air condition system according to claim 17, further comprising a filtering passage in the air purification device, wherein the plasma generation module is disposed within the filtering passage.

19. The air condition system according to claim 18, wherein a cross section of the filtering passage is a slit, and the plasma generation module is in a stripe shape; or
the cross section of the filtering passage is circular, and the plasma generation module is in a ring shape; or
the cross section of the filtering passage is square, and the plasma generation module is in a curved shape.

20. The air condition system according to claim 17, further comprising a carrier member, wherein filter holes are opened on a side wall of the carrier member, and the plasma generation module is disposed on the carrier member.
